(19) United States
(12) Patent Application Publication
(10) Pub. No.: US 2024/0238454 A1
(43) Pub. Date: Jul. 18, 2024

(54) LUNG LESION LOCALIZATION MULTI-IMAGING CONTRAST AGENT COMPOSITION FOR RESTRICTIVE LUNG RESECTION

(71) Applicant: KOREA UNIVERSITY RESEARCH AND BUSINESS FOUNDATION, Seoul (KR)

(72) Inventors: Hyun Koo KIM, Seoul (KR); Yuhua QUAN, Seoul (KR); Ji Yun RHO, Seoul (KR); Byeong Hyeon CHOI, Seoul (KR); Ji Ho PARK, Daejeon (KR); Woo Jin BACK, Daejeon (KR)

(21) Appl. No.: 18/276,311

(22) PCT Filed: Feb. 9, 2022
(86) PCT No.: PCT/KR2022/095025

§ 371 Date: Aug. 8, 2023

(30) Foreign Application Priority Data
Feb. 9, 2021 (KR) 10-2021-0018457
Feb. 8, 2022 (KR) 10-2022-0016515

(51) Int. Cl. A61K 49/04; A61K 47/36; A61K 49/00

(52) U.S. Cl. CPC A61K 49/0438; A61K 47/36; A61K 49/0032; A61K 49/0076; A61K 2123/00

(57) ABSTRACT

The present invention relates to a multi-imaging contrast agent composition and a method for preparing same, in which the composition comprises a hydrogel formed by crosslinking a first dextran polymer and a second dextran polymer, wherein the hydrogel is loaded with a fluorescent dye and a contrast material. The composition comprises a dextran-based hydrogel in which a fat-soluble contrast material and a water-soluble fluorescent dye are simultaneously loaded, and has the advantage of enabling accurate localization of lung lesions.

### TECHNICAL FIELD

The present disclosure relates to a lung lesion localization multi-imaging contrast agent composition for restrictive lung resection and a method for preparing the same.

### BACKGROUND ART

The most cancer occurring in people in Korea is stomach cancer, colon cancer, lung cancer, thyroid cancer, and liver cancer. Based on national cancer incidence statistics, the risk of developing cancer is increasing, with the lifetime probability of developing cancer analyzed as 1 in 3 for men and 1 in 4 for women and the like. In particular, in the case of lung cancer, the number of lung cancer patients is expected to increase as air pollution worsens due to industrialization and the number of smokers increases, especially among adolescents and women. In the United States, lung cancer is also the most common cause of cancer-related death for both men and women.

The main types of lung cancer are small cell lung cancer (SCLC) and non-small cell lung cancer (NSCLC). The small cell lung cancer (SCLC) is a fast-growing type of lung cancer. The SCLC spreads much faster than non-small cell lung cancer.

There are three different types of small cell lung cancer: small cell carcinoma (oat cell cancer), mixed small cell/large cell carcinoma, and mixed small cell carcinoma. Most small cell lung cancer is an oat cell type. The non-small cell lung cancer (NSCLC) is the most common type of lung cancer. There are three types of NSCLC: adenocarcinoma, squamous cell carcinoma, and large-cell carcinoma. The adenocarcinoma mainly occurs in the peripheral part of the lung, also occurs frequently in women or non-smokers, and often metastasizes even if the size is small, and recently, the frequency of its occurrence has been increased. Next, the squamous cell carcinoma is mainly found in the center of the lung, causes symptoms which mainly grow into the bronchial lumen to block the bronchial tubes, and is common in men and known to be closely related to smoking. The large-cell carcinoma mainly occurs near the lung surface (peripheral lung), and about a half thereof occurs in the large bronchial tubes. The large-cell carcinoma accounts for about 4 to 10% of all lung cancers, has generally large cell sizes, and some of the cells tend to rapidly proliferate and metastasize, so that it is known to have a worse prognosis than other non-small cell lung cancers. Conventional treatments for lung cancer include palliative care, surgery, chemotherapy and radiotherapy.

If lung cancer is diagnosed early, the patient may be recovered through drug treatment and radiation therapy, but if the symptom of disease has worsened to a certain level, surgical resection is essential, and drug treatment and radiation therapy are combined. However, the early diagnosis rate of lung cancer is not high at about 20%. Accordingly, most lung cancer patients undergo lung resection, but the lung has a characteristic of functioning smoothly due to its structural characteristics, and thus when performing lung resection, it is advantageous to cut the lung at an appropriate distance from the tumor, but with minimal resection by accurately identifying the location and size of the lesions.

Meanwhile, among imaging technologies, near-infrared fluorescence imaging technology is attracting attention as non-invasive disease diagnosis and monitoring technology, but may perform ideal biometric imaging due to an advantage that the near-infrared region of 700 to 900 nm has low background autofluorescence and low light scattering to enable imaging of deep tissue. Among fluorescent dyes for fluorescent imaging, indocyanine green (ICG) is a near-infrared fluorescent dye approved for use through clinically and is mainly used for staining liver blood vessels and the heart, and applied to various diagnostic and imaging technologies.

Since it was known in 1999 that intravenous infusion of ICG before surgery makes it possible to detect the interface of liver cancer under near-infrared fluorescence imaging during surgery, recently, a cancer detection method using passive targeting fluorescent contrast agents that can be used in actual clinical practice have been actively used.

However, when injecting a fluorescent contrast agent through intravenous injection, a high concentration of at least 5 mg/kg of fluorescent contrast agent needs to be injected to detect cancer, and uniformly applied to various cancers such as colon cancer, breast cancer, skin cancer, and lung cancer, but in the case of lung cancer, there is a problem that the detection rate is very low. Specifically, it has been reported that in lung cancer, cancer tissue is mainly located at a depth of 1 cm or more from the lung surface, so that the cancer detection rate is only 10%.

Non-patent prior art related to the present disclosure includes Onda N, Kimura M, Yoshida T, Shibutani M. Preferential tumor cellular uptake and retention of indocyanine green for in vivo tumor imaging. Int J Cancer. 2016 Aug. 1; 139(3):673-82.

### DISCLOSURE OF THE INVENTION

### Technical Goals

An aspect of the present disclosure is to provide a multi-imaging contrast agent composition in which a water-soluble fluorescent dye and a fat-soluble contrast material are simultaneously loaded to have an excellent lung lesion labeling effect.

Another aspect of the present disclosure is to provide a method for preparing the composition.

Yet another aspect of the present disclosure is to provide a hydrogel loaded with the fluorescent dye and the contrast material.

However, technical goals to be achieved are not limited to those described above, and other goals not mentioned above are clearly understood by one of ordinary skill in the art from the following description.

### Technical Solutions

According to an embodiment, the present disclosure provides a multi-imaging contrast agent composition including a hydrogel formed by crosslinking a first dextran polymer and a second dextran polymer, in which in the first dextran polymer, hydroxyl groups are substituted with vinyl sulfone groups, and in the second dextran polymer, hydroxyl groups are substituted with vinyl sulfone groups and thiol groups.

In the first dextran polymer, 10 to 30 mol % of the hydroxyl groups may be substituted with vinyl sulfone groups, and in the second dextran polymer, 10 to 30 mol % of the hydroxyl groups may be substituted with thiol groups and vinyl sulfone groups.

Further, the present disclosure provides a multi-imaging contrast agent composition including a hydrogel formed by crosslinking a first dextran polymer and a second dextran polymer, in which the hydrogel is loaded with a fluorescent dye and a contrast material.

In an embodiment of the present disclosure, in the first dextran polymer, hydroxyl groups may be substituted with vinyl sulfone groups, and in the second dextran polymer, hydroxyl groups may be substituted with thiol groups and vinyl sulfone groups.

In another embodiment of the present disclosure, in the first dextran polymer, 10 to 30 mol % of the hydroxyl groups may be substituted with vinyl sulfone groups, and in the second dextran polymer, 10 to 30 mol % of the hydroxyl groups may be substituted with thiol groups and vinyl sulfone groups.

In yet another embodiment of the present disclosure, the fluorescent dye may be a cyanine-based fluorescent dye.

In yet another embodiment of the present disclosure, the fluorescent dye may be at least one selected from the group consisting of Indocyanine green (ICG), Alexa Fluor 647, Alexa Fluor 660, Alexa Fluor 680, Alexa Fluor 700, Alexa Fluor 780, cy3.5, cy5, cy5.5, cy7, Cypate, ITCC, NIR820, NIR2, IRDye78, IRDye80, IRDye82, IRDye680, IRDye700, IRDye800, DiD, DiR, Cresyl Violet, Nile Blue, Oxazine 750, Rhodamine 800, Texas Red, and mixtures thereof.

In yet another embodiment of the present disclosure, the contrast material may be iodized oil.

In yet another embodiment of the present disclosure, the contrast material may be included in less than 30 vol % with respect to the hydrogel. When the contrast material is included in 30 vol % or more, an unstable emulsion is formed (FIG. 8), and the phases may be separated, making it unsuitable for administration into the body.

In yet another embodiment of the present disclosure, the fluorescent dye may be included at a concentration of 0.005 mg/mL to 0.02 mg/mL. If the concentration is out of the range, there may be a problem that the fluorescence intensity decreases.

In yet another embodiment of the present disclosure, the composition may be for labeling lung lesions.

In yet another embodiment of the present disclosure, the hydrogel may be formed by mixing the first dextran polymer and the second dextran polymer and then gelating the mixture within 20 seconds to 250 seconds. When the time exceeds the range, there may be a problem that clinical application is difficult, and the gelation may be performed within desirably 20 seconds to 250 seconds, more desirably 20 seconds to 180 seconds, and most desirably 30 seconds to 40 seconds, which enables rapid gelation when used clinically, and thus it may be usefully used.

According to another embodiment, the present disclosure also provides a method for preparing a multi-imaging contrast agent composition including: preparing a first composition including a first dextran polymer in which hydroxyl groups are substituted with vinyl sulfone groups; a contrast material and a fluorescent dye (S1);
sonicating the first composition (S2);
preparing a second composition including a second dextran polymer in which hydroxyl groups are substituted with thiol groups and vinyl sulfone groups; a contrast material and a fluorescent dye (S3);
sonicating the second composition (S4); and
mixing the first and second compositions that have been sonicated and gelating the mixture (S5).

### Effects

According to the present disclosure, it is possible to solve a problem of the low detection rate of lung cancer with conventional fluorescent contrast agents used in cancer detection, and the multi-imaging contrast agent of the present disclosure includes a hydrogel simultaneously loaded with a fat-soluble contrast material and a water-soluble fluorescent dye. The hydrogel gelates at a rapid rate to be easily used clinically, and has an effect of remaining in the lesion area of lung cancer for a long time.

The effects of the present disclosure are not limited to the aforementioned effects, and other aspects, which are not mentioned above, will be clearly appreciated by a person having ordinary skill in the art from the following description.

### BRIEF DESCRIPTION OF DRAWINGS

FIG. 1 illustrates a schematic diagram of a hydrogel in which a dextran-based hydrogel is loaded with ICG and iodized oil.

FIG. 2A illustrates a method of synthesizing a precursor by substituting dextran and a method of synthesizing a hydrogel.

FIG. 2B illustrates a method of loading ICG and iodized oil into a hydrogel.

FIG. 3 illustrates a nuclear magnetic resonance analysis spectrum of a dextran polymer substituted with vinyl sulfone groups.

FIG. 4 illustrates a nuclear magnetic resonance analysis spectrum of a dextran polymer substituted with vinyl sulfone groups and thiol groups.

FIG. 5 illustrates a result of confirming the gelation time according to gel concentration and degree of functional group substitution.

FIG. 6 illustrates in-vivo imaging results of gels containing 10% and 20% iodized oil.

FIG. 7 illustrates a result of confirming the gelation time of hydrogels containing different amounts of iodized oil.

FIG. 8 illustrates the stability of hydrogels containing different amounts of iodized oil with CT signals.

FIG. 9 illustrates a result of confirming the fluorescence intensity according to an ICG concentration.

FIG. 10 illustrates a result of confirming the release time of ICG and iodized oil.

FIG. 11 illustrates a dual syringe used when applying a contrast agent of the present disclosure.

FIG. 12A illustrates a process of confirming a multi-imaging contrast agent in an in-vivo dog model.

FIG. 12B shows photographs visible to the naked eye depending on a concentration of iodized oil.

FIG. 12C illustrates fluorescence measured depending on a concentration of iodized oil.

FIG. 12D illustrates the degree of colocalization when the concentration of iodized oil is 10% and 20%.

FIG. 13A illustrates a process of confirming a multi-imaging contrast agent in an in-vivo rabbit lung cancer model.

FIG. 13B illustrating labeling a lesion area of a rabbit lung cancer model with a contrast agent.

FIG. 13C illustrates a CT scan result that a contrast material and ICG are maintained at the lesion area for 24 hours and later.

FIG. 13D is an ex-vivo image of photographing the fluorescence of a contrast agent.

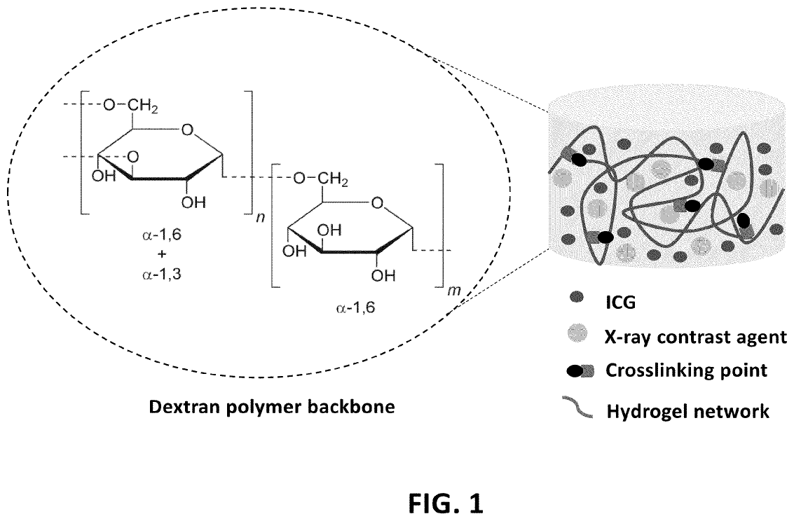

### BEST MODE FOR CARRYING OUT THE INVENTION

As the number of patients with early lung nodules increases due to an increase in computed tomography (CT) screening, the need for restrictive lung resection increases, and thus, the importance of localization of lung lesions, which identifies and labels the lesion location before surgery, has also increased. Therefore, localization studies are actively being conducted to pre-label the location of lung lesions using iodized oil, hookwire, or the like as a radiocontrast, under CT guidance before surgery. However, lung lesion localization methods that have been currently used suffer from difficulties such as causing complications such as pain and pneumothorax, the inconvenience of using devices, and difficulty in detecting lung resection margins.

To reduce these difficulties, studies have recently been conducted on localization of lung lesions guided by fluorescence imaging using the fluorescent properties of indocyanine green (ICG). The ICG is an FDA-approved material and is known as a contrast agent that has no radiation exposure and may easily identify the location of lung lesions using fluorescent imaging surgical equipment during thoracoscopic surgery. When localizing lung nodules using ICG, it is possible to easily identify the location of lung lesions in real time through near-infrared fluorescence thoracoscopy during surgery.

However, when ICG, a water-soluble material, is used alone for localization in lung lesions, there are disadvantages in that the ICG spreads easily to surrounding lung tissue, making it difficult to accurately determine the location of lung lesions and identify lung resection margins, and when injected to a depth of 1 cm or deeper, it is difficult to identify the location of lung lesions during surgery due to the low penetration of ICG.

On the other hand, iodized oil, an X-ray contrast agent mainly used in clinical practice, is a lipophilic material, which has a limitation that it is not easy to be loaded on the water-soluble ICG and the hydrogel.

Therefore, as a result of extensive research to develop a contrast agent which possibly overcomes the limitations of materials previously used for localization of lung lesions in the related art, and may simultaneously load ICG and iodized oil, a hydrogel type received attention.

The hydrogel is a material that may contain a large amount of water after a water-soluble polymer forms a 3D structure through chemical covalent bonds. Because of these characteristics, hydrogels have been applied and used in the fields of tissue engineering, regenerative medicine, diagnostics, and drug delivery for many years. In particular, “injectable” hydrogels made by modifying physical and chemical properties of organic chemical functional groups or polymers are considered clinically important.

Therefore, the present disclosure provides a multi-imaging contrast agent composition including a hydrogel formed by crosslinking a first dextran polymer and a second dextran polymer, in which the hydrogel is loaded with a fluorescent dye and a contrast material.

In the present disclosure, in the first dextran polymer, hydroxyl groups are substituted with vinyl sulfone groups, and in the second dextran polymer, hydroxyl groups are substituted with vinyl sulfone groups and thiol groups. In the first dextran polymer, 10 to 30 mol % of the hydroxyl groups are substituted with vinyl sulfone groups, and in the second dextran polymer, 10 to 30 mol % of the hydroxyl groups are substituted with vinyl sulfone groups and thiol groups. The thiol groups and the hydroxyl groups of the first dextran polymer and the second dextran polymer may be crosslinked to each other to form a hydrogel. That is, 10 to 30 mol % of the hydroxyl group of the hydrogel may be substituted. In the present disclosure, “the hydroxyl groups are substituted with the vinyl sulfone groups and the thiol groups” specifically means that vinyl sulfone groups are substituted for hydroxyl groups, and then thiol groups are added to double-bonded carbon portions of the vinyl sulfone groups. A desirable example thereof is shown in Chemical Formula 1 below.

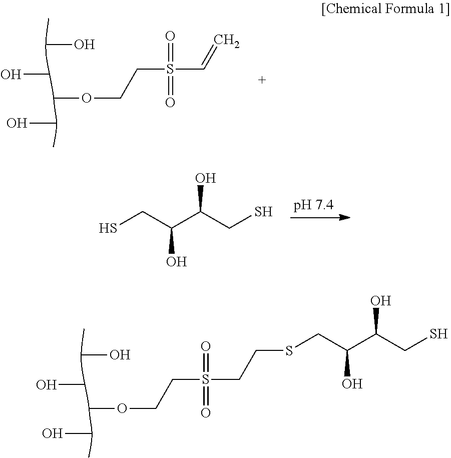

The fluorescent dye of the present disclosure is a water-soluble dye and may be a cyanine-based fluorescent dye. The fluorescent dye may be at least one selected from the group consisting of Indocyanine green (ICG), Alexa Fluor 647, Alexa Fluor 660, Alexa Fluor 680, Alexa Fluor 700, Alexa Fluor 780, cy3.5, cy5, cy5.5, cy7, Cypate, ITCC, NIR820, NIR2, IRDye78, IRDye80, IRDye82, IRDye680, IRDye700, IRDye800, DiD, DiR, Cresyl Violet, Nile Blue, Oxazine 750, Rhodamine 800, Texas Red, and mixtures thereof. The contrast material of the present disclosure is a fat-soluble contrast material, and may be desirably iodized oil.

In more detail, the composition of the present disclosure includes ICG as a water-soluble dye capable of performing CT imaging, and iodized oil as a fat-soluble contrast material, which is an X-ray contrast agent, and has advantages of easily determining the location of lung lesions at a deep location and enabling maintaining for a long time.

The contrast material may be included in less than 30 vol % with respect to the hydrogel. Desirably, the contrast material may be included in 0.01 to less than 30 vol %. When the content of the contrast material is 30 vol % or more, an unstable emulsion form with separated phases is formed so that a problem may be caused that it is difficult to be used.

The fluorescent dye is included in the hydrogel at a concentration of 0.005 mg/mL to 0.02 mg/mL, and the best intensity value is confirmed in the concentration range.

The present disclosure also provides a method for preparing a multi-imaging contrast agent composition including preparing a first composition including a first dextran polymer in which hydroxyl groups are substituted with vinyl sulfone groups; a contrast material and a fluorescent dye (S1);
sonicating the first composition (S2);
preparing a second composition including a second dextran polymer in which hydroxyl groups are substituted with thiol groups and vinyl sulfone groups; a contrast material and a fluorescent dye (S3);
sonicating the second composition (S4); and
mixing the first and second compositions that have been sonicated and gelating the mixture (S5).

The gelating in step S5 of the present disclosure may be performed within 20 seconds to 250 seconds, desirably 20 seconds to 180 seconds, and more desirably 30 seconds to 40 seconds, which enables rapid gelation when used clinically, and thus it may be usefully used.

The contrast agent composition of the present disclosure may further include known contrast materials and fluorescent dyes in addition to the aforementioned contrast material and fluorescent dye as the active ingredient, and may be used in combination with other known diagnostic methods to obtain specific information about lung disease lesions.

Meanwhile, when the fluorescent contrast agent of the present disclosure is a near infra-red fluorescent dye, the fluorescent contrast agent exhibits fluorescence characteristics in a near-infrared wavelength region. Therefore, the composition of the present disclosure may acquire images through a near-infrared fluorescence imaging (NIR imaging) method known to those skilled in the art, but is not limited thereto, and for example, may photograph images using a near-infrared optical image measuring system (NIR optical imaging system) and also measure a near-infrared fluorescence spectrum using a fluorescence spectrophotometer.

The terms used in the embodiments are used for the purpose of description only, and should not be construed to be limited. The singular expression includes the plural expression unless the context clearly dictates otherwise. In the present disclosure, it should be understood that term “including” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which embodiments pertain. Terms which are defined in a generally used dictionary should be interpreted to have the same meaning as the meaning in the context of the related art, and are not interpreted as ideal or excessively formal meanings unless otherwise defined in the present disclosure.

In addition, in the description with reference to the accompanying drawings, like components designate like reference numerals regardless of reference numerals and a duplicated description thereof will be omitted. In describing the embodiments, a detailed description of related known technologies will be omitted if it is determined that they unnecessarily make the gist of the embodiments unclear.

Hereinafter, Examples will be described in detail with reference to the accompanying drawings. However, since various modifications may be made to Examples, the scope of the present disclosure is not limited or restricted by these Examples. It should be understood that all modifications, equivalents and substitutes for Examples are included in the scope of the present disclosure.

### Mode for Carrying Out the Invention

### Example 1. Preparation Method of Hydrogel

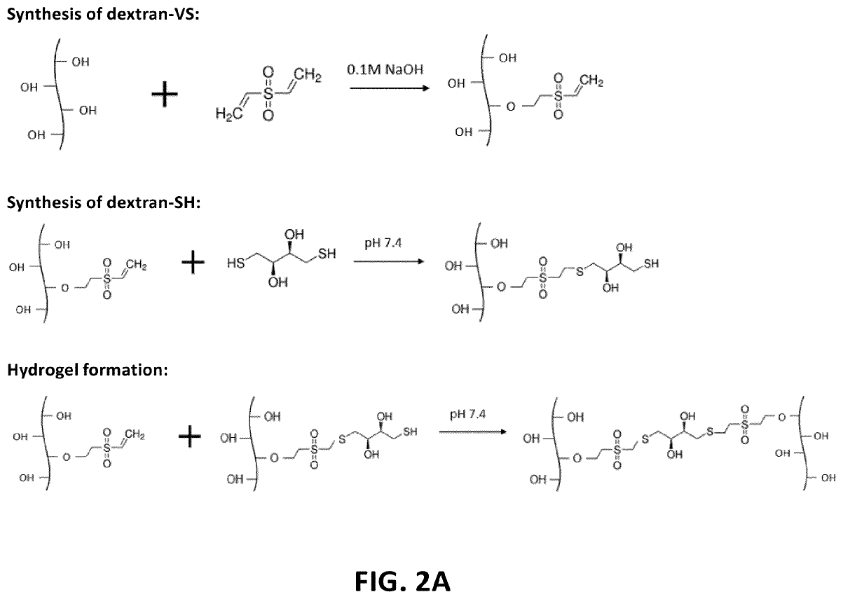

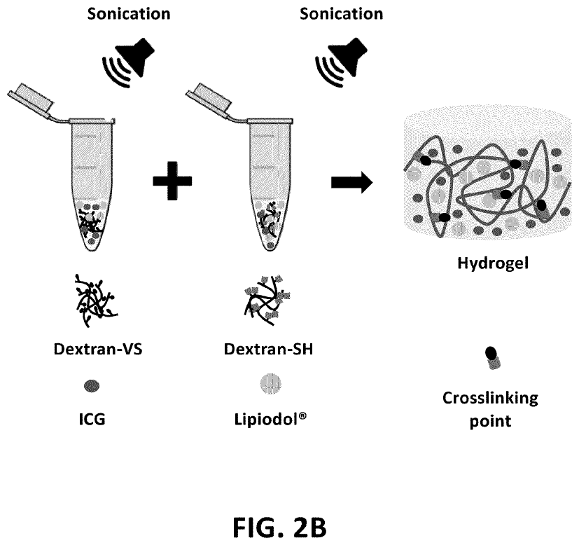

In Example, for the synthesis of a hydrogel, as illustrated in FIG. 2A, hydroxyl functional groups of a dextran polymer were modified to a polymer having vinyl sulfone groups (Precursor 1) of Chemical Formula 2 below and thiol groups (Precursor 2) of Chemical Formula 3 below using click chemistry.

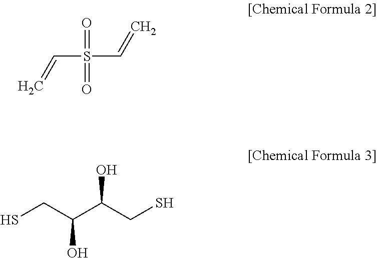

More specifically, divinylsulfone (MW: 118.16 g/mol) 1.25 times greater than the number of moles of hydroxyl groups included in the dextran polymer was added in 0.1 M NaOH (to pH 11 or higher), and then the synthesis was performed with different reaction times depending on a desired degree of substitution of vinyl sulfone groups. When the chemical reaction was terminated according to the reaction time, 6 M HCl was added to lower the pH to 7 or less. The dextran polymer substituted with the vinyl sulfone groups was dialyzed with water for about 3 days (3.5 kDa molecular weight cut-off dialysis membrane) to remove chemicals that were not consumed during the reaction. After dialysis, a first dextran polymer in solid form substituted with vinyl sulfone groups was obtained through freeze-drying. Whether substitution with the vinyl sulfone groups has been performed well was confirmed through nuclear magnetic resonance (1H NMR) analysis.

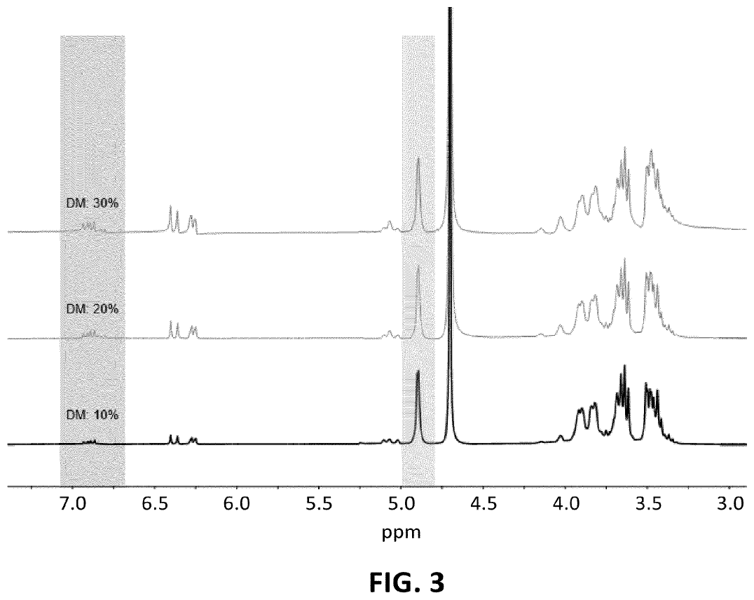

FIG. 3 illustrated 1H NMR spectra of dextran polymers substituted with 10 mol %, 20 mol %, and 30 mol % of vinyl sulfone groups, respectively. Here, the degree of substitution was confirmed by comparing after integrating areas under curve of the peaks corresponding to 8=6.9 and at 8=2.

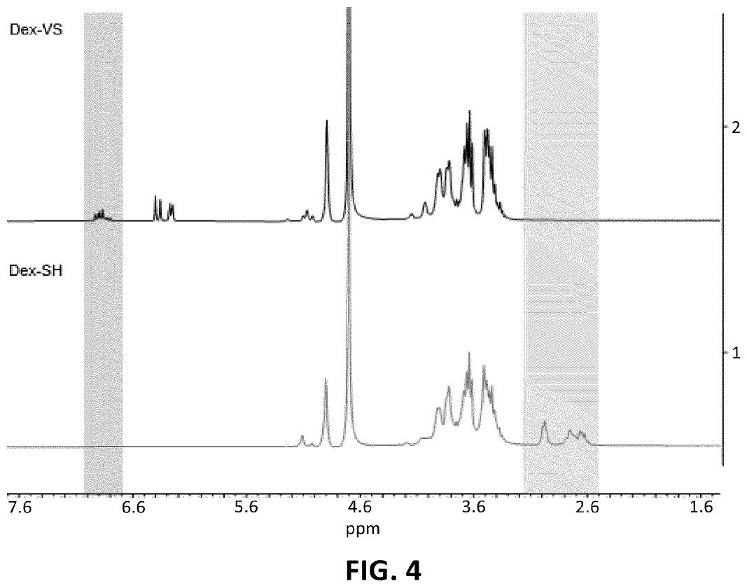

To synthesize the second dextran polymer substituted with vinyl sulfone groups and thiol groups, the first dextran polymer substituted with 10 mol %, 20 mol %, and 30 mol % of vinyl sulfone groups was adjusted to pH to 7.4 using a 1 M phosphate buffer saline. Dithiothreitol (MW: 154.253 g/mol) was added in at least 5 times greater than the number of moles of substituted vinyl sulfone groups, and the reaction proceeded for about 40 minutes so that thiol groups were added to all the vinyl sulfone groups. After 40 minutes, 1 M HCl was added to make the pH a slightly acidic condition, and dithiothreitol that was not consumed during the reaction was removed through dialysis for 3 days. After dialysis, the second dextran polymer substituted with the thiol and vinyl sulfone groups was freeze-dried. Successful substitution of thiol groups was confirmed by whether the 8=6.9 peak disappeared using nuclear magnetic resonance (1H NMR) (FIG. 4).

Next, the prepared freeze-dried first and second dextran polymers were each dissolved in PBS, and then added with ICG and iodized oil, respectively. Thereafter, sonication was performed for 5 minutes to emulsify the fat-soluble iodized oil. After sonication, equal volumes of the obtained first dextran polymer solution and the second dextran polymer solution were mixed to be gelated. The mixing conditions were pH 7.4 (in vivo pH) and room temperature, and gel formation was completed after about 30 to 40 seconds (FIG. 2B).

### Example 2. Optimization of Gel Concentration and Degree of Functional Group Substitution

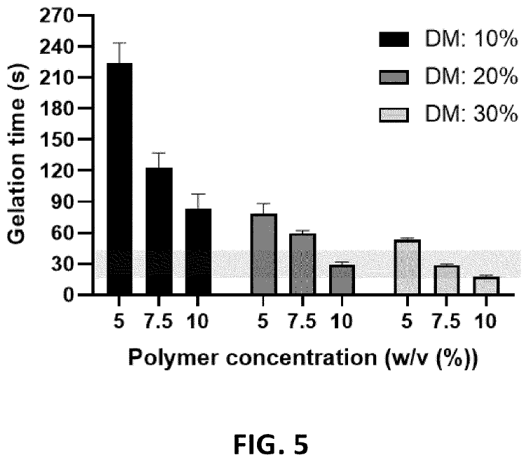

In order to optimize the gelation time in the method, the gelation time according to the concentration of the polymer and the degree of functional group substitution was confirmed and illustrated in FIG. 5. The concentration of the polymer was represented by w/v % with respect to PBS, and the degree of functional group substitution (DM) was 10, 20, and 30 mol %, respectively.

As a result of the experiment at a polymer concentration of 10% (w/v), it was confirmed that a hydrogel with the degree of functional group substitution of the first dextran polymer and the second dextran polymer of 20 mol % had the gelation time of about 30 seconds which is clinically optimized.

### Example 3. Optimization of Hydrogel According to Content of Iodized Oil

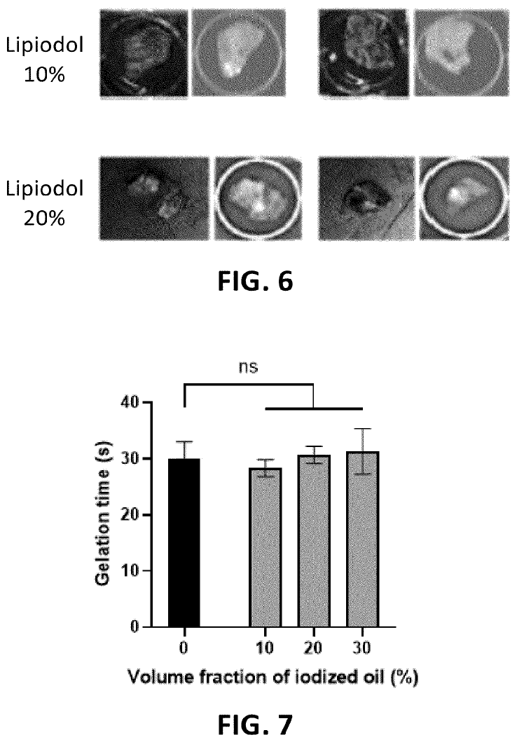

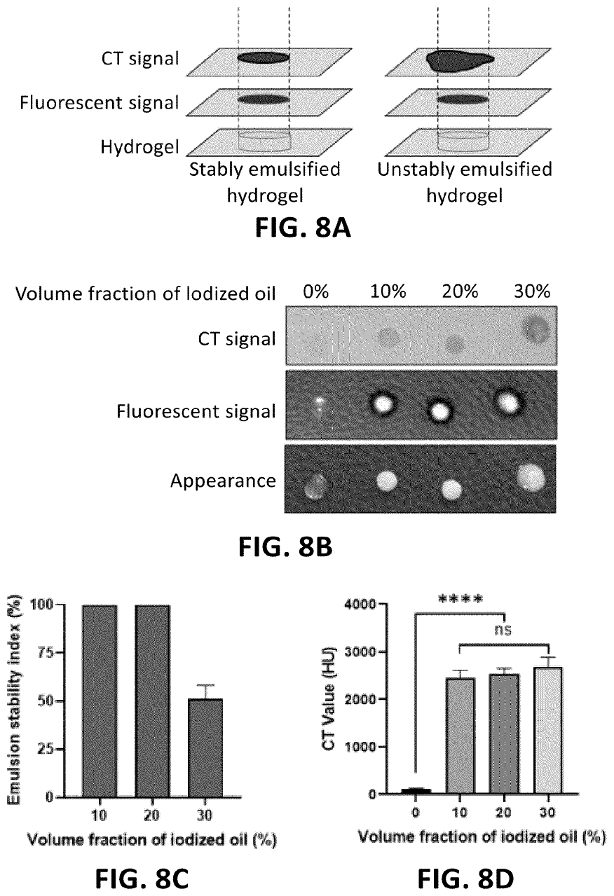

The physical properties of the hydrogel were confirmed according to the content of iodized oil. The iodized oil was experimented at 0, 10, 20, and 30% (v/v) based on the total volume of the gel, respectively, and it was confirmed that if the content was 30% (v/v) or more, an uneven emulsion occurred (FIGS. 6 and 8). In addition, when the content was less than 20% (v/v), it was confirmed that the CT signal was not confirmed depending on the volume of the gel according to the in vivo experiment result (FIG. 6). Accordingly, the iodized oil concentration of 20% (v/v) was set as an optimized concentration. It was confirmed that when 20% (v/v) of iodized oil was contained, the gelation time was maintained at about 30 seconds without change (FIG. 7).

### Example 4. Optimization of ICG Concentration

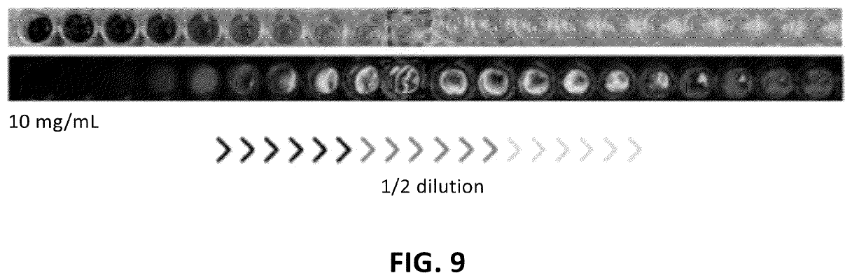

In addition, in order to set the ICG concentration, an optimization experiment was directly performed while referring to the literature of “Minimally Invasive Electro-Magnetic Navigational Bronchoscopy-Integrated Near-Infrared-Guided Sentinel Lymph Node Mapping in the Porcine Lung (PLOS ONE 10(5):e0126945. https://doi.org/10.1371/journal.pone.0126945)” of Hironobu Wada et al. At this time, it was confirmed that the best intensity value was shown when the ICG concentration was about 0.005 mg/ml to 0.02 mg/ml (FIG. 9).

### Example 5. Confirmation of Fluorescence Performance of Hydrogel According to Concentrations of ICG and Iodized Oil

From the results, it may be expected that the hydrogel containing 0.01 mg/ml of ICG and 20% iodized oil will have the most effective fluorescence performance. Accordingly, after synthesizing a hydrogel formulation loaded with 0.01 mg/ml of ICG and 20% iodized oil, the release time of ICG and iodized oil was confirmed in phosphate buffered saline (PBS) at 37° ° C. for 24 hours.

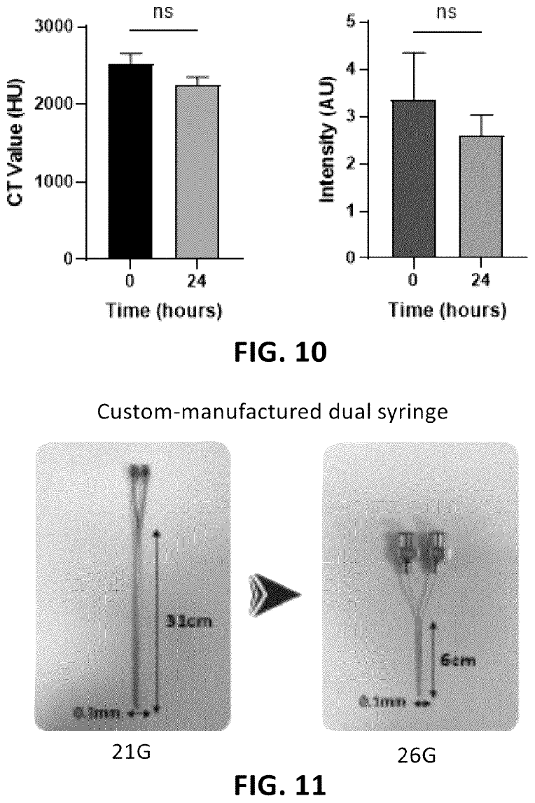

As a result, it was secured that ICG and iodized oil were successfully loaded into the hydrogel for 24 hours (FIG. 10).

### Example 6. Confirmation of In-Vivo Effect of Multi-Imaging Contrast Gel

The efficacy of the multi-imaging contrast gel in the optimized formulation was also confirmed through in-vivo experiments (dog and rabbit lung cancer models). After injection into the lung, a dual syringe was fabricated and used in in-vivo experiments in order to form a hydrogel by meeting the first dextran polymer solution and the second dextran polymer solution containing ICG and iodized oil (FIG. 11).

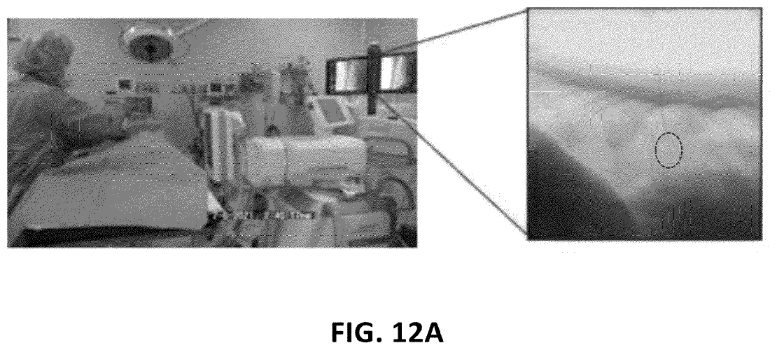

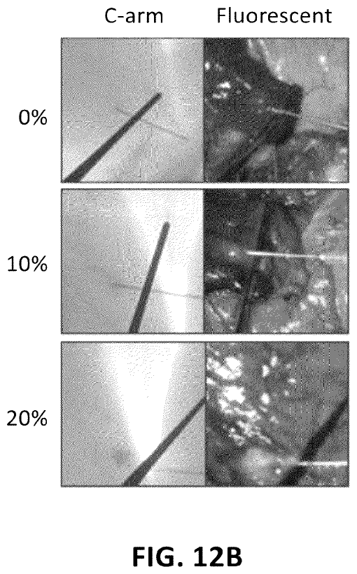

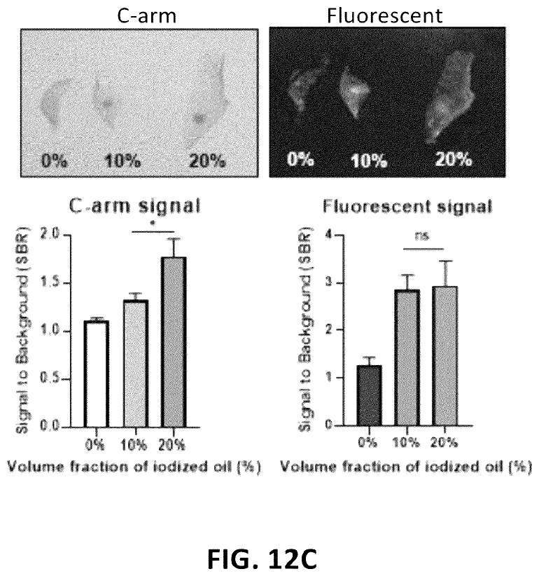

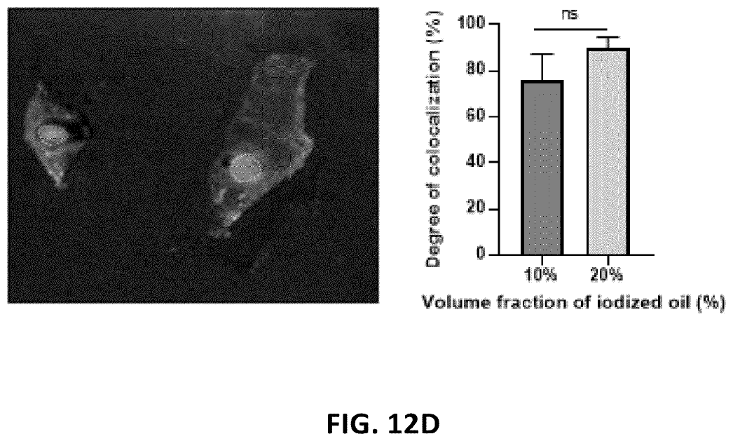

Specifically, an in vivo experiment was performed using a hydrogel containing 0.01 mg/ml of ICG and loaded with 0%, 10%, or 20% of iodized oil. The in-vivo experiment using dogs was performed (FIG. 12A), and photography and fluorescence comparison according to a concentration of iodized oil were illustrated in FIGS. 12B and 12C, respectively. As a result of the experiment, compared to a control group (a formulation formed in an emulsion only with iodized oil and ICG without a hydrogel), it was confirmed that when using a multi-imaging contrast gel, iodized oil and ICG were well maintained in the injected lung tissue (FIGS. 12B and 12C). In addition, similarly to the results of in-vitro experiments, a hydrogel containing 10% iodized oil was almost not visible during C-arm imaging, and a gel containing 20% iodized oil showed an ICG signal and a CT signal capable of identifying a lesion area while maintained well in the lesion area (FIG. 12D).

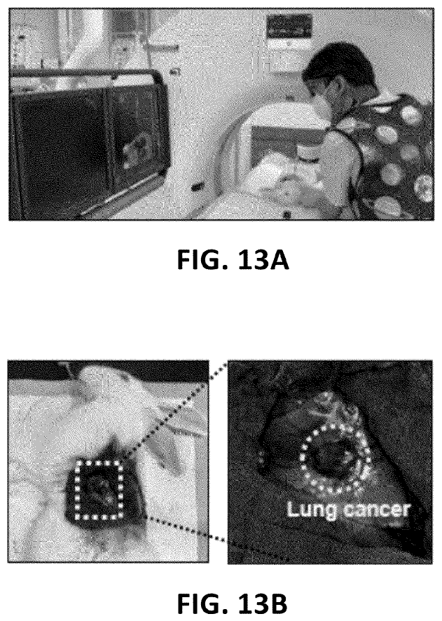

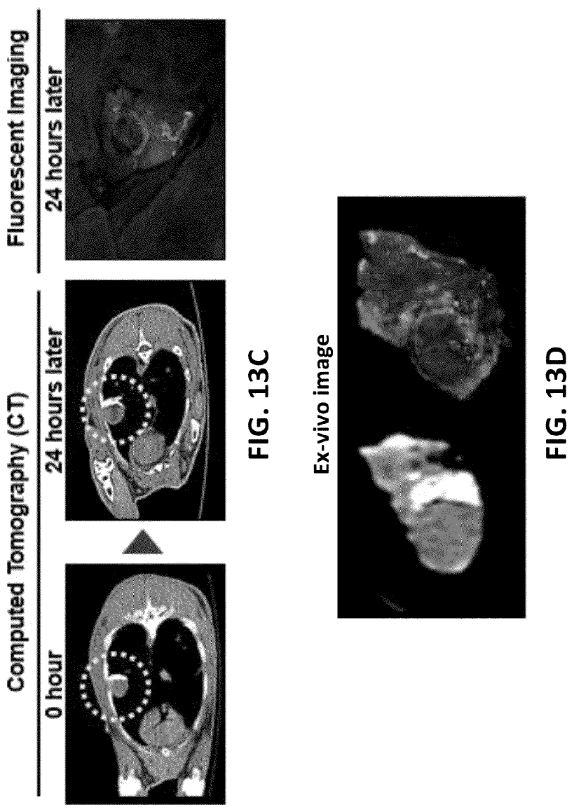

Through an additional in-vivo optimization step using dogs, the effect of the multi-imaging contrast gel in a rabbit lung cancer model was confirmed (FIG. 13A). As a result of the experiment, it may be confirmed in FIG. 13B that the lesion area of rabbit lung cancer is well labeled, and it was confirmed that iodized oil and ICG were well maintained in the lesion area of lung cancer for 24 hours (FIG. 13C). The ex-vivo image was shown in FIG. 13D.

As described above, although the embodiments have been described by the restricted drawings, various modifications and variations can be applied on the basis of the embodiments by those skilled in the art. For example, even if the described techniques are performed in a different order from the described method, and/or components such as a system, a structure, a device, a circuit, and the like described above are coupled or combined in a different form from the described method, or replaced or substituted by other components or equivalents, an appropriate result can be achieved.

Therefore, other implementations, other embodiments, and equivalents to the appended claims fall within the scope of the claims to be described below.

### INDUSTRIAL APPLICABILITY

The present disclosure relates to a multi-imaging contrast agent composition and a preparation method capable of localizing lung lesions when applying medical technology such as lung resection, and more specifically, a fluorescent dye and a contrast material are loaded into a dextran-based hydrogel to have excellent lung lesion labeling ability and in vivo retention ability. Therefore, the multi-imaging contrast agent composition of the present disclosure and the preparation method thereof may be applied in various ways for the purpose of labeling lesion areas in intracorporeal resection, etc.

1. A multi-imaging contrast agent composition comprising:
a hydrogel formed by crosslinking a first dextran polymer and a second dextran polymer,
wherein the hydrogel is loaded with a fluorescent dye and a contrast material.

2. The multi-imaging contrast agent composition of claim 1, wherein in the first dextran polymer, hydroxyl groups are substituted with vinyl sulfone groups, and in the second dextran polymer, hydroxyl groups are substituted with vinyl sulfone groups and thiol groups.

3. The multi-imaging contrast agent composition of claim 2, wherein in the first dextran polymer, 10 to 30 mol % of the hydroxyl groups are substituted with vinyl sulfone groups, and in the second dextran polymer, 10 to 30 mol % of the hydroxyl groups are substituted with thiol groups and vinyl sulfone groups.

4. The multi-imaging contrast agent composition of claim 1, wherein the fluorescent dye is a cyanine-based fluorescent dye.

5. The multi-imaging contrast agent composition of claim 4, wherein the fluorescent dye is at least one selected from the group consisting of Indocyanine green (ICG), Alexa Fluor 647, Alexa Fluor 660, Alexa Fluor 680, Alexa Fluor 700, Alexa Fluor 780, cy3.5, cy5, cy5.5, cy7, Cypate, ITCC, NIR820, NIR2, IRDye78, IRDye80, IRDye82, IRDye680, IRDye700, IRDye800, DiD, DiR, Cresyl Violet, Nile Blue, Oxazine 750, Rhodamine 800, Texas Red, and mixtures thereof.

6. The multi-imaging contrast agent composition of claim 1, wherein the contrast material is iodized oil.

7. The multi-imaging contrast agent composition of claim 1, wherein the contrast material is comprised in less than 30 vol % with respect to the hydrogel.

8. The multi-imaging contrast agent composition of claim 1, wherein the fluorescent dye is comprised at a concentration of 0.005 mg/mL to 0.02 mg/mL.

9. The multi-imaging contrast agent composition of claim 1, wherein the composition is for labeling lung lesions.

10. The multi-imaging contrast agent composition of claim 1, wherein the hydrogel is formed by mixing the first dextran polymer and the second dextran polymer and then gelating the mixture within 20 seconds to 250 seconds.

11. A method for preparing a multi-imaging contrast agent composition comprising:
preparing a first composition comprising a first dextran polymer in which hydroxyl groups are substituted with vinyl sulfone groups; a contrast material and a fluorescent dye (S1);
sonicating the first composition (S2);
preparing a second composition comprising a second dextran polymer in which hydroxyl groups are substituted with thiol groups and vinyl sulfone groups; a contrast material and a fluorescent dye (S3);
sonicating the second composition (S4); and
mixing the first and second compositions that have been sonicated and gelating the mixture (S5).

12. The method for preparing the multi-imaging contrast agent composition of claim 11, wherein in the first dextran polymer, 10 to 30 mol % of the hydroxyl groups are substituted with vinyl sulfone groups, and in the second dextran polymer, 10 to 30 mol % of the hydroxyl groups are substituted with thiol groups and vinyl sulfone groups.

13. The method for preparing the multi-imaging contrast agent composition of claim 11, wherein the fluorescent dye is a cyanine-based fluorescent dye.

14. The method for preparing the multi-imaging contrast agent composition of claim 13, wherein the fluorescent dye is at least one selected from the group consisting of Indocyanine green (ICG), Alexa Fluor 647, Alexa Fluor 660, Alexa Fluor 680, Alexa Fluor 700, Alexa Fluor 780, cy3.5, cy5, cy5.5, cy7, Cypate, ITCC, NIR820, NIR2, IRDye78, IRDye80, IRDye82, IRDye680, IRDye700, IRDye800, DiD, DiR, Cresyl Violet, Nile Blue, Oxazine 750, Rhodamine 800, Texas Red, and mixtures thereof.

15. The method for preparing the multi-imaging contrast agent composition of claim 11, wherein the contrast material is iodized oil.

16. The method for preparing the multi-imaging contrast agent composition of claim 11, wherein the contrast material is comprised in less than 30 vol % with respect to the hydrogel.

17. The method for preparing the multi-imaging contrast agent composition of claim 11, wherein the fluorescent dye is comprised at a concentration of 0.005 mg/mL to 0.02 mg/mL.

18. The method for preparing the multi-imaging contrast agent composition of claim 11, wherein the gelating in the S5 is performed within 20 seconds to 250 seconds.

19. A hydrogel formed by crosslinking a first dextran polymer in which hydroxyl groups are substituted with vinyl sulfone groups and a second dextran polymer in which hydroxyl groups are substituted with vinyl sulfone groups and thiol groups,
wherein in the first dextran polymer, 10 to 30 mol % of the hydroxyl groups are substituted with vinyl sulfone groups, and in the second dextran polymer, 10 to 30 mol % of the hydroxyl groups are substituted with thiol groups and vinyl sulfone groups.
